(19) United States
(12) Patent Application Publication
(10) Pub. No.: US 2016/0333396 A1
(43) Pub. Date: Nov. 17, 2016

(54) COMPOSITIONS AND METHODS FOR DETERMINATION OF NUCLEIC ACID AMPLIFICATION STATUS AND KIT FOR PERFORMING SUCH METHODS

(71) Applicants: Rex M. Bitner, Cedarburg, WI (US); Tonny M. Johnson, Madison, WI (US)

(72) Inventors: Rex M. Bitner, Cedarburg, WI (US); Tonny M. Johnson, Madison, WI (US)

(73) Assignee: Rex M. Bitner, Grafton, WI (US)

(21) Appl. No.: 14/714,186

(22) Filed: May 15, 2015

(51) Int. Cl. C12Q 1/68

(52) U.S. Cl. CPC C12Q 1/6816

(57) ABSTRACT

Compositions, methods and kits for determination of the nucleic acid amplification status of nucleic acid samples, and analysis of other high copy nucleic acid products, are disclosed, as well as a matrix and method for storage of nucleic acid amplification products. In a preferred embodiment, the method provides for a determination of whether or not a nucleic acid amplification reaction has produced an anticipated product, or not, and provides, without the use of electricity, a visual readout detectable with the unaided human eye.

### FIELD OF THE INVENTION

This invention relates to methods of purifying one or more nucleic acid products produced by a nucleic acid amplification reaction, and a determination of the nucleic acid amplification status of nucleic acid samples produced by amplification reactions, and also to compositions and kits for use in performing such methods, and providing a matrix and method for storage of nucleic acid amplification products.

### BACKGROUND OF THE INVENTION

The amplification of deoxyribonucleic acid (DNA) or ribonucleic acid (RNA) plays an important role in scientific procedures, particularly in molecular diagnostics. There are a number of known methods for amplifying single stranded and double stranded DNA or RNA contained in biological samples such as human blood, serum, urine, cerebral spinal fluid, stool samples, human and animal tissue, cultured cells, plant materials, food, environmental samples, and other specimens. Several of these use isothermal methods for amplifying DNA (see for example Shikata, U.S. Pat. No. 8,697,400 (2014), Hutchinson et al., U.S. Pat. No. 8,497,069 (2013); or RNA (see for example Kurn et al., U.S. Pat. No. 7,846,666 (2010); Siva et al., U.S. Pat. No. 8,399,222 (2013); Yotoriyama et al., U.S. Pat. No. 8,632,998 (2014)) or both DNA and RNA (see for example Notomi et al., U.S. Pat. No. 6,410,278 (2002); Yonekawa et al., U.S. Pat. No. 8,557,523 (2013); Brentano et al., U.S. Pat. No. 8,512,955 (2013); Hoser et al., U.S. Pat. No. 7,824,890 (2010); Jenison et al., U.S. Pat. No. 8,637,250 (2014); Rabbini et al., U.S. Pat. No. 8,445,664 (2013)), and do not require the use of electricity to perform these nucleic acid amplifications. Currently, the evaluation of such isothermal amplification products uses sophisticated, complicated separation and detection systems, such as the use of laser dyes and spectrophotometric detection (such as molecular beacons), laser dyes and electrophoretic separations and spectrophotometric detection (e.g. CODIS profiling), or electrophoretic separations followed by visual or spectrophotometric detection to determine the status of the resultant amplification products. In some cases indications that amplification reactions have taken place are provided, such as the production of turbidity, or the binding of DNA to a dye such as PicoGreen, EvaGreen, etc., but these reaction indicators show positive results when artifacts such as amplification of primer dimers have occurred, but the production of the desired amplification product has not occurred. Thus, there is no indicator of the presence of an amplification product that is of the desired molecular weight or size, as opposed to artifacts such as primer dimer that are not of the desired molecular weight or size. As a result, the utilization of these simple isothermal amplification methods is not coupled to an equivalently simple detection technology for the desired amplification product, thus limiting their application, utility, and economy of use. Known methods of nucleic acid purification that provide for selective purification based upon the molecular weight of nucleic acids are described in: Bitner U.S. Pat. No. 8,519,119 (2013); Bitner U.S. Pat. No. 8,222,397 (2012), Bitner et al., U.S. Pat. No. 8,658,360 (2014); Nargessi U.S. Pat. No. 6,855,499 (2005); Hawkins U.S. Pat. No. 5,898,071 (1999); and Hawkins U.S. Pat. No. 5,705,628 (1998), McKernan et al., U.S. Pat. No. 6,534,262 (2003), Kojima et al., U.S. Pat. No. 7,241,572 (2007), Taylor et al., J. ChromatographyA 890:159-166 (2000); Ahn et al., Biotechniques 29:466-468 (2000); Scott Jr et al., Lett. Appl. Microbiol., 31:95-99 (2000); Lin et al., Biotechniques 29:460-466 (2000); Smith et al., U.S. Pat. No. 6,027,945 (2000); Mrazek et al., Acta Univ. Palacki. Olomuc. Fac. Med. 142:23-28 (1999). Additional methods for nucleic acid purification are described in: Fabis et al., U.S. Patent Application No. 20130158247 (2013); RITT et al., U.S. Patent Application No. 2012/0283426 (2012); Jiang at al., U.S. Patent Application No. 2011/0097782 (2011); Fonnum et al., U.S. Patent Application No. 2010/0207051 (2010); Fredix et al., U.S. Patent Application No. 2010/0036109; Himmelreich et al., U.S. Patent Application No. 2012/0130061 (2012); Himmelreich et al., U.S. Patent Application No. 2011/0224419 (2011).

However, these methods do not allow the determination of the status of a nucleic acid amplification product, nor do they provide information about the molecular weight of the amplification product. Moreover, they do not provide a visual indicator discernible using the unaided human eye. Additionally, these methods do not provide a method coupled with a matrix for the long term storage of the amplification products, to facilitate later follow-up evaluations, if desired.

### SUMMARY OF THE INVENTION

The present invention relates to compositions, methods and kits for the determination of whether or not a nucleic acid amplification has produced amplification products consistent with a positive result, or whether such amplification products have not been produced. In one aspect, the present invention provides this determination in a visual format. In one embodiment, the method does not require the use of electricity, or complicated, costly molecules such as laser dyes, bioluminescent molecules, or molecular beacons. In a preferred embodiment, the result may be discerned with normal, unassisted, human vision.

In another aspect, the compositions and methods are selected so they provide a stable visual record of the results. In a preferred embodiment, the compositions and methods also provide for the stable storage of the nucleic acid amplification products, to facilitate later follow-up evaluations, if desired.

In another aspect, the presence of naturally occurring nucleic acid products may be present in sufficiently high copy number that nucleic acid amplification is not required for the present invention to be practiced. Examples of such situations may include, but are not limited to, viral infections such as influenza, certain rhinoviruses, porcine epidemic diarrhea virus, blue tongue bovine virus, foot and mouth disease virus, and norovirus.

In another aspect, the invention relates to kits for use in determining whether or not a nucleic acid amplification reaction has produced nucleic acid amplification products, or if no nucleic acid amplification products were formed. The kit comprises, a binding matrix, a binding solution with a formulation and one or more reporter molecules, as described.

### DEFINITIONS

All of the compositions and methods disclosed and claimed herein may be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

“Nucleic acid amplification products” are any nucleic acid products produced by a nucleic acid amplification reaction. These include but are not limited to RNA, DNA, chemical derivatives of RNA, chemical derivatives of DNA, RNA incorporating nucleotides not normally found in naturally occurring RNA, DNA incorporating nucleotides not normally found in naturally occurring DNA, and combinations of the aforementioned.

The term “target polynucleotide” is used herein to refer to particular nucleic acids to be detected in the methods described herein. Target nucleic acids include, for example, loci of interest (e.g., single nucleotide polymorphisms) in genotyping studies, mRNAs of interest in expression studies, as well as non-coding RNAs. Target polynucleotides that are originally (i.e., prior to experimental intervention) found in the form of RNA are also termed “target RNAs” herein.

As used herein, the term “complementary” refers to the capacity for pairing between two nucleotides, i.e., if a nucleotide at a given position of a nucleic acid is capable of hydrogen bonding with a nucleotide of another nucleic acid, then the two nucleic acids are considered to be complementary to one another at that position. Complementarity (Watson-Crick or non-canonical pairing) between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands and the consequent stacking interactions.

“Hybridization” refers to the binding of a nucleic acid to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated. In particular embodiments, hybridizations are carried out under stringent hybridization conditions. The phrase “stringent hybridization conditions” generally refers to a temperature in a range from about 5° C., to about 20° C., or 25° C., below the melting temperature (Tm) for a specific sequence at a defined ionic strength and pH. As used herein, the Tm is the temperature at which a population of double-stranded nucleic acid molecules becomes half-dissociated into single strands. Methods for calculating the Tm of nucleic acids are well known in the art (see, e.g., Berger and Kimmel (1987) METHODS IN ENZYMOLOGY, VOL. 152: GUIDE TO MOLECULAR CLONING TECHNIQUES, San Diego: Academic Press, Inc. and Sambrook et al. (1989) MOLECULAR CLONING: A LABORATORY MANUAL, 2ND ED., VOLS. 1-3, Cold Spring Harbor Laboratory), both incorporated herein by reference). As indicated by standard references, a simple estimate of the Tm value may be calculated by the equation: Tm=81.5+0.41(% G+C), when a nucleic acid is in aqueous solution at 1 M NaCl (see, e.g., Anderson and Young, Quantitative Filter Hybridization in NUCLEIC ACID HYBRIDIZATION (1985)). The melting temperature of a hybrid (and thus the conditions for stringent hybridization) is affected by various factors such as the length and nature (DNA, RNA, base composition) of the primer or probe and nature of the target nucleic acid (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art.

“Stringent conditions” or “high stringency conditions,” for example, can be hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 mg/ml), 0.1% SDS, and 10% dextran sulfate at 42° C., with washes at 42° C. in 0.2% SSC (sodium chloride/sodium citrate) and 50% formamide at 55° C., followed by a wash with 0.1× SSC containing EDTA at 55° C. By way of example, but not limitation, it is contemplated that buffers containing 35% formamide, 5×SSC, and 0.1% (w/v) sodium dodecyl sulfate (SDS) are suitable for hybridizing under moderately non-stringent conditions at 45° C. for 16-72 hours. Furthermore, it is envisioned that the formamide concentration may be suitably adjusted between a range of 20-45% depending on the probe length and the level of stringency desired. Additional examples of hybridization conditions are provided in several laboratory manual known for a person skilled in the art. Similarly, “stringent” wash conditions are ordinarily determined empirically for hybridization of a target to a probe, or a probe derived amplicon. The amplicon/target are hybridized (for example, under stringent hybridization conditions) and then washed with buffers containing successively lower concentrations of salts, or higher concentrations of detergents, or at increasing temperatures until the signal-to-noise ratio for specific to non-specific hybridization is high enough to facilitate detection of specific hybridization. Stringent temperature conditions will usually include temperatures in excess of about 30° C., more usually in excess of about 37° C., and occasionally in excess of about 45° C. Stringent salt conditions will ordinarily be less than about 1.0 M, usually less than about 500 mM, more usually less than about 150 mM (Wetmur et al., 1966, J. Mol. Biol., 31:349-370; Wetmur, 1991, Critical Reviews in Biochemistry and Molecular Biology, 26:227-259). As used herein, the term “target nucleic acid molecules” and “target nucleic acid sequences” are used interchangeably and refer to molecules or sequences from a target genomic region to be studied. The pre-selected probes determine the range of targeted nucleic acid molecules. Thus, the “target” is sought to be sorted out from other nucleic acid sequences. A “segment” is defined as a region of nucleic acid within the target sequence, as is a “fragment” or a “portion” of a nucleic acid sequence.

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules, but double-stranded oligonucleotides, or oligonucleotides that are partially single-stranded and partially double-stranded may also be produced.

A “capture probe” or “probe” is a nucleic acid capable of binding to a target nucleic acid of complementary sequence through one or more types of chemical bonds, generally through complementary base pairing, usually through hydrogen bond formation (but also through co-ordinal metal complexes), thus forming a duplex structure. The probe binds or hybridizes to a “capture probe binding site.” Capture probe or probe includes any form of a nucleic acid probe selected from DNA, RNA, mRNA, complementary DNA (cDNA; e.g., a reverse-transcribed copy of an mRNA); ribosomal RNA; short interfering RNA (siRNA); a ribozyme; transfer RNA (tRNA); spliced mRNA; a cDNA copy of a splice mRNA; unspliced mRNA; a cDNA copy of an unspliced mRNA; microRNA, an oligonucleotide, an aptmer (DNA or RNA), a non-cording RNA, DNA or RNA molecules produced synthetically or by amplification and the like.

Capture probes or probe can vary significantly in size. Generally, capture probes or probes are at least 6 nucleotides in length up to the full length target polynucleotide. The capture probe or probe may be perfectly complementary to the target nucleic acid sequence or may be less than perfectly complementary. In certain embodiments, the capture probe has at least 65% identity to the complement of the target nucleic acid sequence over a sequence of at least 7 nucleotides, more typically over a sequence in the range of 10-30 nucleotides, and often over a sequence of at least 14-25 nucleotides, and more often has at least 75% identity, at least 85% identity, at least 90% identity, or at least 95%, 96%, 97%, 98%, or 99% identity. It will be understood that certain bases (e.g., the 3′ base of a primer) are generally desirably perfectly complementary to corresponding bases of the target nucleic acid sequence. Capture probe or probe typically anneal most specifically to the target sequence under stringent hybridization conditions.

“Metal ion and metal oxide” or “Metal ions and metal oxides” are selected from the group consisting of iron, copper, gallium, cobalt, nickel, calcium, zinc, cadmium, silver, gold, zirconium, hafnium, titanium, palladium, platinum, aluminum, vanadium, lead, manganese, tin and ruthenium.

The term “enzyme” as used herein refers to a protein molecule produced by living organisms, or through chemical modification of a natural protein molecule, that catalyzes chemical reaction of other substances without itself being destroyed or altered upon completion of the reactions. Examples of other substances, include, but are not limited to chemiluminescent, chromogenic and fluorogenic substances or protein-based substrates.

The term “complexing” or “complex” as used herein refers to the association of two or more molecules, usually by non-covalent bonding, e.g., with a metal ion-chelator and a metal ion complexed with (i.e., noncovalently bound to) a protein or, for instance, of an antibody and antigen, enzyme and enzyme substrate, ligand and receptor (e.g. biotin and avidin), nucleic acid and its complementary strand, a protein with another protein or with a nucleic acid having affinity for the first protein, and the like.

The term “amplified polynucleotide” means the product of copying the polynucleotide, wherein the product has a nucleotide sequence that is the same as or complementary to at least a portion of the nucleotide sequence of the polynucleotide. An amplified polynucleotide can be produced by any of a variety of amplification methods that use the nucleic acid, or an amplicon thereof, as a template including, for example, polymerase extension, polymerase chain reaction (PCR), rolling circle amplification (RCA), nucleic acid sequenced based amplification (NASBA), transcription mediated amplification (TMA), strand displacement amplification (SDA), loop-mediated isothermal amplification (LAMP), RNA based single primer isothermal amplification (ribo-SPIA), signal mediated amplification of RNA (SMART), recombinase polymerase amplification (RPA), helicase dependent amplification (HAD), ligation extension, in vitro RNA replication using replicases or ligation chain reaction. An amplified polynucleotide can be a polynucleotide molecule having a single copy of a particular polynucleotide sequence (e.g. a PCR product) or multiple copies of the polynucleotide sequence (e.g. a concatameric product of RCA).

“Nucleic acid” may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

### DETAILED DESCRIPTION OF THE PREFERRED EMBODIMENTS

In one aspect, the invention relates to a method of co-purifying one or more reporter molecules and the products of a nucleic acid amplification to a binding matrix, wherein said reporter molecule, in the absence of the nucleic acid amplification products, has substantially no binding affinity for the binding matrix under the binding conditions used, and when the nucleic acid amplification product(s) are present, then the nucleic acid amplification product(s) and the reporter molecule both co-purify to the binding matrix. The co-purification can be achieved by the direct binding of the reporter molecule to the binding matrix, or by the reporter molecule becoming associated in a complex with one or more of the nucleic acid amplification products, wherein the amplification product-reporter molecule complex binds to the binding matrix. The copurification may be achieved using purification methods comprising PEG and alcohol and salt, glycerol, 1,2 propane diol, 1,3 propane diol, or one of the methods described by Bitner U.S. Pat. No. 8,519,119 (2013); Bitner U.S. Pat. No. 8,222,397 (2012), Bitner et al., U.S. Pat. No. 8,658,360 (2014); Nargessi U.S. Pat. No. 6,855,499 (2005); Hawkins U.S. Pat. No. 5,898,071 (1999); and Hawkins U.S. Pat. No. 5,705,628 (1998), McKernan et al., U.S. Pat. No. 6,534,262 (2003); Jiang at al., U.S. Patent Application No. 2011/0097782 (2011); Himmelreich et al., U.S. Patent Application No. 2011/0224419 (2011). The nucleic acid amplification products may comprise DNA, RNA, chemically modified DNA, chemically modified RNA, or a combination of any of the above. The method of the present invention provides for the co-purification of a reporter molecule with these amplification products based on the molecular weight or size of the amplification product. For example, changing the concentration of PEG, 1,2 propane diol, glycerol, alcohol, salt, or combinations thereof in the aforementioned methods changes the precipitation or complexing of the amplification products based on the products size and molecular weight. Thus the binding solutions of the present invention may be adjusted to the purification of the desired molecular weight or size of the amplification product coupled to the co-purification of the reporter molecule, thus differentiating between the desired product, artifacts such as primer dimer, and reactions in which no product is produced, thereby providing a simple determination of whether or not the target nucleic amplification products were formed, or not formed.

In another aspect of the invention, the nucleic acid amplification product is purified and bound to a binding matrix prior to the addition of the reporter molecule to the binding matrix. In one embodiment of this method, using an appropriate amount of polyethylene glycol (PEG) and salt, or 1,2 propane diol and salt, to allow for the purification of the desired size of the amplification product, but not the purification of smaller nucleic acids, the nucleic acid amplification product is then adsorbed to a binding matrix. The reporter molecule is then added to the binding matrix, and binds to the nucleic acid adsorbed on the binding matrix. For example, the reporter molecule may be bound to a nucleic acid capture probe which specifically binds to the nucleic acid amplification product, by mechanisms such as hybridization based on sequence complementarity, or triple helix binding. The reporter molecule thereby binds specifically to the nucleic acid amplification product, if formed; in the absence of the nucleic acid amplification product, binding of the reporter molecule does not occur. In another embodiment of this method, the amplified nucleic acid product may be hybridized to the binding matrix based upon the complementarity of the amplification product sequence to the sequence of an immobilized oligonucleotide on a specific area of the binding matrix. The reporter molecule is then added, and binds to the amplification product based upon the presence of a nucleotide sequence that is present in the amplification product, but is not the same as, or complementary to, the sequence used to immobilize the amplification product.

Enzymatic reporter molecules may by selected by those of skill in the art. Suitable examples, without limitation, may be selected from the group consisting of alkaline phosphatase, glucose-6-phosphate dehydrogenase, catalase, horseradish peroxidase, glucose oxidase, glucose dehydrogenase, NADH oxidase, uricase, urease, creatininase, sarcosine oxidase, xanthine oxidase, creatinase, creatine kinase, creatine amidohydrolase, cholesterol esterase, cholesterol oxidase, glycerol kinase, hexokinase, glycerol-3-phosphate oxidase, lactate dehydrogenase, alanine transaminase, aspartate transaminase, amylase, lipase, esterase, gammaglutamyl transpeptidase, L-glutamate oxidase, pyruvate oxidase, diaphorase, bilirubin oxidase, laccase, tyrosinase, and their mixtures.

Reporter molecules that are not enzymes may also be chosen by those skilled in the art. Examples of such reporter molecules include labeled anti DNA or anti RNA antibodies labeled with colloidal gold. A method of colloidal gold labeling of antibodies is described in Georghegan, U.S. Pat. No. 4,880,751 (1989). This method may also be used for the labeling of proteins and peptides, other than antibodies, that bind to the target nucleic acid, or to the reporter molecule that copurifies with the target nucleic acid.

In order to amplify the signal of the reporter molecules, wherein the reporter molecule is a polypeptide or polynucleotide or a small molecule, said reporter molecules may be labeled with or conjugated with detection molecules selected from the group consisting of enzyme, polypeptide, polynucleotide, nanoparticles and nanobeads (colored beads or particles including colloidal gold, cellulose nanobeads, latex beads, silver enhanced gold nanoparticles, blue nanoparticles, and carbon black nanoparticles).

In another embodiment, the reporter molecule is bound to an affinity molecule that has affinity for nucleic acids, such that when the reporter molecule-affinity molecule is added, it binds to the amplification product that has previously been bound to the matrix. The addition of this combined reporter molecule-affinity molecule may take place during the purification of the nucleic acid amplification product in a copurification, or may be added in a two-step process wherein the nucleic acid amplification product is first purified and bound to a binding matrix, and the reporter molecule-affinity molecule composite is added after the purification of the nucleic acid amplification product, and binds to the purified nucleic acid. One example of such an affinity molecule is a zirconium particle, as zirconium is known to have affinity for both nucleic acids and certain reporter molecules, particularly those containing one or more phosphate groups Bitner et al., EP 0391608 (1990), such as, for example, an alkaline phosphatase, such as bovine alkaline phosphatase. When a reporter molecule has affinity for a different material, such as a metal oxide that does not have substantial affinity for nucleic acids, a combined material comprising that material with a second material that has a high affinity for the nucleic acid target, would provide a suitable composite material for binding to one or more reporter molecules, and have affinity for a target nucleic acid, specifically a product of a nucleic acid amplification. In one aspect of this embodiment, a nucleic acid amplification reaction in solution is combined with one of the above mentioned formulations, such as PEG and NaCl such that the larger molecular weight nucleic acids (such as the desired product) form a complex of nucleic acids in solution; this complex binds to a binding matrix, such as cellulose, Nargessi U.S. Pat. No. 6,855,499 (2005), or cellulose with functionally charged groups, Hawkins U.S. Pat. No. 5,705,628 (1998). The reporter molecule bound to an affinity molecule that has affinity for nucleic acids is then added, and binds to the nucleic acid complex previously bound to the matrix. In the case of no amplification product being present, the nucleic acid complex does not form in solution, is not bound to the matrix, and the reporter molecule bound to an affinity molecule that has affinity for nucleic acids does not bind to the matrix. Alternatively, the reporter molecule may be added to the nucleic acid mixture and copurify with the nucleic acid in a one-step procedure. Thus in this embodiment, the presence or absence of the nucleic acid amplification product may be determined.

In another embodiment, the reporter molecule may not have an affinity to the nucleic acid amplification product but may simply be physically entangled, or entrapped, with the nucleic acid during the purification process, such that the reporter molecule and nucleic acid amplification product copurify. One example of this embodiment is the attachment of the reporter molecule to a particle small enough to be in suspension in the binding solution formulation (such as PEG and salt, 1,2 propane diol and salt, or glycerol and salt, as previously disclosed), which although it does not have an affinity for nucleic acids, may become physically entangled with the nucleic acid during the purification process. During this copurification process, the reporter-particles may become physically entangled with each other, and form agglomerates that fall out of suspension in the solution. In the absence of nucleic acid amplification products, this agglomeration would not take place, and the particles would not fall out of suspension in the solution. Some particle materials suitable for this embodiment may be considered to be binding matrices as they facilitate the copurification of reporter molecule and nucleic acid amplification products. Thus, the presence or absence of the nucleic acid amplification product may be determined.

In a preferred embodiment, one or more reporter molecules may be detected using a device that does not require electricity, such as an optical filter that reduces certain wavelengths of light, or accentuates the visibility of particular wavelengths of light. In a more preferred embodiment, the one or more reporter molecules provide a visual readout using the normal, unassisted, human eye. Optionally, the one or more reporter molecules may also be detected or quantified with an electronic device, or the visual readout may be documented or transmitted via devices such as a camera or cell phone.

One embodiment is a method of detecting and/or quantitating a nucleic acid amplification product comprising a reporter molecule having an enzymatic activity, with or without a label, for visual detection together or simultaneously with an affinity molecule having affinity for said nucleic acid amplification product thereby forming a complex comprising said affinity molecule and said reporter molecule thereby forming a reporter complex comprising said nucleic acid amplification product and said affinity molecule and said reporter molecule, and detecting and/or quantitating said nucleic acid amplification product using assays specific for said reporter molecule; wherein the presence of said reporter molecule in said reporter complex indicates the presence of said nucleic acid amplification product.

In one embodiment, the binding matrix and binding conditions are selected from materials and methods that provide for the stable storage of the visually observable results. The nucleic acid amplification products bound to the binding matrix may also be stably stored on the binding matrix, thus providing a means for further analysis of the nucleic acid amplification products if desired.

Any number of known binding matrices may be used in the foregoing methods, depending upon the type of nucleic acid amplification products, the type of reporter molecule being used, and the binding conditions being used. Those skilled in the art will be able to select binding matrices that are compatible with the foregoing methods. Examples of suitable binding matrices include, but are not limited to, cellulose, cellulose modified with functional chemical groups, nitrocellulose, cellulose acetate, nylon, poly vinyl difluoride (PVDF), pectin, metal oxides, silica, chemically modified silica, or combinations thereof. The binding matrix may, for example, be in any of a variety of forms, such as, without limitation, particles, paramagnetic particles, membranes, coatings, sheets or monolithic bodies.

Any number of known binding conditions may be used in the foregoing methods, depending upon the type of nucleic acid amplification products, the type of reporter molecule being used, and the binding matrix being used. Those skilled in the art will be able to select binding conditions that are compatible with the foregoing methods. Examples of suitable binding conditions include, but are not limited to, methods involving polyalkyene glycol and salt including those described by Bitner U.S. Pat. No. 8,222,397 (2012), Nargessi U.S. Pat. No. 6,855,499 (2005); Hawkins U.S. Pat. No. 5,898,071 (1999); and Hawkins U.S. Pat. No. 5,705,628 (1998) which are hereby incorporated in their entirety by reference, methods comprising 1,2 propane diol such as those described by Bitner U.S. Pat. No. 8,222,397 (2012), Himmelreich et al., U.S. Patent Application No 2011/0224419 (2011), the purification conditions of which are hereby incorporated in their entirety by reference, methods involving the use of alcohol precipitation of nucleic acids, methods of binding nucleic acids based on the pH of the binding solution such as those described by Smith et al., U.S. Pat. No. 6,806,362 (2004); Baker U.S. Pat. No. 6,718,742 (2004); Baker U.S. Pat. No. 6,914,137 (2005); and Fabris et al., U.S. Patent Application US 2013/0158247 which are hereby incorporated in their entirety by reference.

In one embodiment of the present invention, the binding matrix and binding conditions are selected from materials and methods that provide for the stable storage of the nucleic acid amplification products, to facilitate later follow-up evaluations, if desired. Those skilled in the art will be able to select binding matrices that are compatible with the foregoing methods that also provide suitable long term storage of the nucleic acid amplification products. Examples of suitable binding matrices and methods include, but are not limited to, those described by Bitner U.S. Pat. No. 8,222,397 (2012), Nargessi U.S. Pat. No. 6,855,499 (2005); Hawkins U.S. Pat. No. 5,898,071 (1999); and Hawkins U.S. Pat. No. 5,705,628 (1998) which are hereby incorporated by reference in their entirety, and those described by Bitner U.S. Pat. No. 8,222,397 (2012), Himmelreich et al., U.S. Patent Application No 2011/0224419 (2011) the entirety of which is incorporated by reference herein. In a preferred embodiment, the binding matrix is cellulose, or cellulose with positively charged groups linked to the cellulose surface.

In another aspect of the invention, the binding matrix may comprise one or more specific locations on the binding matrix where nucleic acid sequences complementary to the desired nucleic acid amplification product have been immobilized on the surface, such that the amplification products bind with specificity to this location, and the reporter molecule-nucleic acid amplification product complex formed using the methods of the present invention also binds specifically to this one or more location. If more than one nucleic acid sequence is subject to amplification, then additional locations specific for the additional product sequences may also be included in one or more specific locations in the binding matrix surface, each with its own complementary nucleic acid molecules immobilized at their specific locations.

Any number of known reporter molecules may be used in the foregoing methods, depending upon the type of nucleic acid amplification products, the type of co-purification method being used, and the binding matrix being used. Those skilled in the art will be able to select reporter molecules that are compatible with the foregoing methods. Examples of suitable reporter molecules include, but are not limited to, alkaline phosphatase, glucose-6-phosphate dehydrogenase, catalase, horseradish peroxidase, glucose oxidase, glucose dehydrogenase, NADH oxidase, uricase, urease, creatininase, sarcosine oxidase, xanthine oxidase, creatinase, creatine kinase, creatine amidohydrolase, cholesterol esterase, cholesterol oxidase, glycerol kinase, hexokinase, glycerol-3-phosphate oxidase, lactate dehydrogenase, alanine transaminase, aspartate transaminase, amylase, lipase, esterase, gammaglutamyl transpeptidase, L-glutamate oxidase, pyruvate oxidase, diaphorase, bilirubin oxidase, laccase, tyrosinase and their mixtures. Those skilled in the art will be able to select substrates for the aforementioned reporter molecules that are compatible with the methods described in this disclosure, thus allowing detection of the reporter molecule.

In one embodiment, the reporter molecule may be attached to a particle, thereby forming a reporter-particle complex. Those skilled in the art will be able to select particles that are compatible with the attachment to one or more reporter molecules to form a reporter-particle complex, and also enable to copurification with the amplification product under the conditions described in the disclosed methods. Examples of suitable particles are macro-beads, micro-beads or nano-beads, both magnetic and non-magnetic, which may be selected from the group consisting of, but not limited to: silica, iron, agarose, Sephadex (GE Healthcare), Sepharose (GE Healthcare), dextran, gelatin, glass, polymer, metal, nitrocellulose, hydrogels, glass, quartz, mica, carbon, apatite, alumina, silica, silicon carbide, silicon nitride, boron carbide, graphite, polycarbonate, polypropylene, polyamide, phenol resin, epoxy resin, polycarbodiimide resin, polyvinyl chloride, polyvinylidene fluoride, polyethylene fluoride, polyimide, and acrylate resin. In a preferred embodiment, the reporter-particle is sufficiently small so as to remain in suspension in the binding solution in the absence of the nucleic acid amplification product, and will become agglomerated, or entangled within the nucleic acid complex formed, thus agglomerating the reporter-particles to a larger size such that the agglomerated reporter-particles no longer remain in suspension. It is not necessary for the particles to have an affinity for the nucleic acids in the nucleic acid complex, the particles may simply become trapped within the confines of the nucleic acid complex. Binding solutions suitable for this embodiment include, but are not limited to PEG/NaCl, 1,2 propane diol/NaCl, glycerol/NaCl, and ethanol/NaCl. For example, an extensive compilation of similar solutions may be found in Himmelreich et al., U.S. Patent Application No 2011/0224419 (2011).

In one embodiment, the one or more reporter molecules are bound to a particle comprising a metal ion or metal oxide, wherein the reporter molecule-metal ion or reporter molecule-metal oxide has an affinity for nucleic acids under the binding conditions used in the present invention, such that the reporter molecule-metal ion-nucleic acid or reporter molecule-metal oxide-nucleic acid has an affinity for the binding matrix under the binding conditions used in the present invention when nucleic acid amplification products are present, but the reporter molecule-metal ion or reporter-metal oxide essentially does not have an affinity for the binding matrix under the binding conditions used in the present invention when the desired nucleic acid amplification products are absent. Those skilled in the art will be able to select metal ions and metal oxides that are compatible with the foregoing methods. Examples of suitable metal ions and metal oxides include, but are not limited to iron, copper, gallium, cobalt, nickel, calcium, zinc, cadmium, silver, gold, hafnium, zirconium, titanium, palladium, platinum, aluminum, vanadium, lead, manganese, tin, ruthenium, and combinations thereof. In a preferred embodiment, the particle size is sufficiently small so as to remain in suspension in the co-purification conditions, such that the reporter molecule-metal ion or reporter molecule-metal oxide exists in suspension under the binding conditions. Such particle sizes may be obtained by those skilled in the art, for example in the case of a metal oxide, by adding metal oxide particles to a solution similar to the binding conditions (in many cases, water would be suitable), and discarding those particles that do not remain in suspension. Such particle suspensions may be combined with reporter molecules so that a metal oxide-reporter molecule complex is formed.

In another embodiment, the particles used to form the reporter molecule-metal ion or reporter molecule-metal oxide particle complex are a colloid, with particles approximately 10 to 10,000 Angstrom in size. Preferably they are between 200 to 5000 Angstroms in size, and more preferably between 600 and 4000 Angstrom in size.

In one embodiment, the reporter molecules are bound to magnetic or paramagnetic particles, such as paramagnetic pectin particles, the making of which is described by Bitner U.S. Pat. No. 8,039,613 (2011). In many of ther binding conditions described, the target nucleic acid will have a binding affinity for pectin, functionally modified pectin, cellulose, functionally modified cellulose, and in particular for pectin or cellulose with positively charged groups covalently bound to their surface. As another example, paramagnetic cellulose particles may also be used, particularly those particles small enough to remain in suspension in the binding conditions used, until an agglomeration with the target nucleic acid amplification product is formed, wherein the agglomeration falls out of suspension. The magnetic or paramagnetic properties of the particle may facilitate subsequent purification of the agglomerated complex, should that be desirable.

In one embodiment, the reporter molecule is bound to a nucleic acid, such that the reporter molecule-nucleic acid construct has sequence complementarity to one or more of the nucleic acid sequences that being amplified. In a preferred embodiment, the complementary sequence above is not complementary to an immobilized nucleic acid sequence present on the surface of the binding matrix wherein the immobilized nucleic acid sequence is complementary to a different nucleic acid sequence also present in the anticipated (e.g. the “positive result”) amplification product, and not complementary to undesired nucleic acid sequences, such as primers, primer dimer, or the sequence in the reporter molecule-nucleic acid construct. The degree of complementarity is selected so that under the binding conditions used, the complementary sequences are sufficient for binding of one molecule to another (e.g. they do not need to be an entirely complementary match), and the non-complementary sequences may have some degree of complementary match, but it is insufficient for the binding of one molecule to another under the binding conditions used.

In another aspect, the invention relates to a composition comprising one or more reporter molecules bound to one or more metal ions or one or more metal oxide molecules such that the resulting reporter molecule-metal ion complex or reporter molecule-metal oxide complex is capable of binding to one or more nucleic acid target molecules under the binding conditions described in the present invention.

In another aspect, the invention relates to a kit for use in determining whether or not a nucleic acid amplification reaction has produced nucleic acid amplification products, or if no nucleic acid amplification products were formed. The kit comprises a binding matrix, a binding solution with a formulation as described above, and a reporter molecule as described above. The binding solution may contain the one or more reporter molecules, or the reporter molecules may be included separately.

In another embodiment, naturally occurring target nucleic acids may be of sufficient quantity in a sample, due to a naturally occurring type of nucleic acid amplification such as certain viral infections, that additional nucleic acid amplification is not required for the present invention to be practiced. In a preferred embodiment, the test material containing the target nucleic acid sequences is combined with a lysing solution that is also suitable as a binding solution, and combined with a binding matrix comprising nucleic acid sequences complementary to the target sequence immobilized to a specific area on the binding matrix surface, so that sufficient numbers of reporter molecules may bind to the target sequences for a reliable result to be obtained. Examples of such target nucleic acids may include, but are not limited to, viral infections such as influenza, papillomavirus, porcine epidemic diarrhea virus, or norovirus.

The following non-limiting examples teach various embodiments of the invention. Only the most preferred embodiments are described in the examples below. However, one skilled in the art of the present invention will be able to use the teachings of the present disclosure to select and use reporter molecules, binding conditions and binding matrices that are within the spirit and scope of the present invention.

### EXAMPLE 1

Zirconium oxide particles (US Research Materials, Inc., Houston, Tex. Catalog Number US7210 ZrO₂ CAS #1314-23-4 45-55 nm particle size) are added to 1 ml of sterile water in a 1.5 ml Eppendorf tube, and are mixed by vortexing for 1 minute. The particles are allowed to settle by gravity for 5 minutes, and the upper 800 ul of solution is added to a second Eppendorf tube containing 200 ul of 0.05 mg/ml of alkaline phosphatase (AP), (Calzyme Laboratories, Inc. San Luis Obisbo Calif., lot 161-14-67 calf intestinal in 50% glycerol 58000 U/ml) thus forming a complex between the AP molecules and the particles of zirconium oxide, the AP-ZrO₂ complex. 60 ul of this solution is added to each of 6, each in duplicate, 1.5 ml Eppendorf tubes containing: (1) 40 ul plasmid DNA, (2) 40 ul PCR 1 DNA, (3) 40 ul PCR 2 DNA, (4) no DNA, (5) 40 ul of a ¼ dilution of PCR 1, and (6) 40 ul of a ¼ dilution of PCR 2. These tubes are mixed by vortexing gently, and to each, 200 ul of 30% PEG 10,000 by weight/30 mM MgCl₂ 50 ul of the corresponding DNA is added, and vortexed gently. Each of the tubes is centrifuged 13,000×g for 1 minute. Each tube is washed with 500 ul of 20% PEG 10,000/20 mM MgCl₂ wash solution, and centrifuged, the supernatants are removed, and the pellets are washed a second time with 500 ul of 20% PEG 10,000/20 mM MgCl₂ solution, and centrifuged. The wash solution is removed and the pellets are resuspended in 120 ul of AP substrate (Moss, Inc. Pasadena, Md., BCIP/NBT Alkaline Phosphatase Substrate Prod # NDTM-100 lot 10278011) and are allowed to react at room temp for 20 minutes. As shown in FIG. 1: the tubes containing plasmid, PCR 1 and PCR 2 DNAs show the particles forming agglomerates between multiple particles such that the suspension of particles falls out of solution, without centrifugation. The products of the AP reaction show strongly colored agglomerated particles at the bottom of the tubes containing DNA, tubes containing ¼ dilutions of PCR 1 and PCR 2 also shows colored, agglomerated particles. The tubes containing no DNA do not show agglutinated particles, and no visible color is formed by AP activity. A tube containing no particles and no DNA, also shows no visible coloration.

### EXAMPLE 2

Zirconium oxide, 10 gm (Sigma-Aldrich, St. Louis, MO USA ZrO₂ Catalog number 230693) are added to 20 ml of sterile water in a 50 ml plastic COREX tube (Corning, Corning, Pa.), and are mixed by vortexing for 3 minutes. The particles are allowed to settle by gravity for 24 hours, and to each of 10 1.5 ml Eppendorf tubes, 1.0 ml of solution is added, each tube is labeled “ZrO₂ particles in suspension”. 800 ul of ZrO₂ in suspension is added to a 1.5 ml Eppendorf tube containing 200 ul of 0.05 mg/ml of alkaline phosphatase (AP), (Calzyme Laboratories, Inc. San Luis Obisbo Calif., lot 161-14-67 calf intestinal in 50% glycerol 58000 U/ml) thus forming a complex between the AP molecules and the particles of zirconium oxide, the AP-ZrO₂ complex.

### EXAMPLE 3

A suspension of AP—ZrO₂ complex particles is prepared as described in Example 2. To each of four tubes, 50 ul of particles is added. To tube 1, 50 ul of PCR 1 DNA is added, to tube 2, 50 ul of PCR 2 DNA is added, to tubes 3 and 4, 50 ul of water containing no DNA is added. The tubes were mixed, 200 ul of binding solution (30% PEG 10,000 and 30 mM MgCl₂) is added per tube, and the tubes are gently vortexed for 3 minutes. Just after vortexing, 200 ul of each solution is slowly pipetted onto a sheet of nitrocellulose so that the liquid is slowly absorbed by the sheet, forming a circle. Outside of these circles, 150 ul of HRP substrate is gently pipetted onto the nitrocellulose sheet, so that the HRP substrate is drawn by capillary action into the regions of the nitrocellulose sheet occupied by the PEG/NaCl mediated complex is formed when DNA is present (in the center of the application spot), as well as the edge of the sample application circle (where the HRP-ZrO₂ complex migrates, in the absence of DNA) The reporter molecule-zirconium oxide particle-nucleic acid is bound to the point of application on the nitrocellulose sheet, in contrast to the reporter molecule-zirconium oxide particles which are located at the edge of the circle produced by the sample application, when nucleic acid is absent from the sample. The difference between these two conditions is easily discernible by the unaided human eye.

Although the present invention has been described in certain specific exemplary embodiments and examples, many additional modifications and variations would be apparent to those skilled in the art, in light of this disclosure. Thus, the exemplary embodiments of the present invention should be considered in all respects to be illustrative, and not restrictive, and the scope of the invention to be determined by any claims supported by this application, and the equivalents thereof, rather than by the foregoing description.

What is claimed:

1. A composition for detecting the presence or absence of the product of a nucleic acid amplification reaction, said composition comprising a reporter molecule that does not have an affinity for said nucleic acid amplification product in a binding solution, that is bound to a metal oxide particle in a first complex, said first complex having an affinity for said nucleic acid amplification product in said binding solution, which may be used for determining the presence or absence of said nucleic acid amplification product utilizing assays specific for said reporter molecule, such that visual detection may be seen by the unassisted human eye.

2. The composition of claim 1, wherein the metal oxide maybe selected from the group consisting of iron, copper, gallium, cobalt, nickel, calcium, zinc, cadmium, silver, gold, hafnium, zirconium, titanium, palladium, platinum, aluminum, vanadium, lead, manganese, tin, ruthenium, and combinations thereof.

3. The composition of claim 1, wherein the reporter molecule may be selected from the group consisting of alkaline phosphatase, glucose-6-phosphate dehydrogenase, catalase, horseradish peroxidase, glucose oxidase, glucose dehydrogenase, NADH oxidase, uricase, urease, creatininase, sarcosine oxidase, xanthine oxidase, creatinase, creatine kinase, creatine amidohydrolase, cholesterol esterase, cholesterol oxidase, glycerol kinase, hexokinase, glycerol-3-phosphate oxidase, lactate dehydrogenase, alanine transaminase, aspartate transaminase, amylase, lipase, esterase, gammaglutamyl transpeptidase, L-glutamate oxidase, pyruvate oxidase, diaphorase, bilirubin oxidase, laccase, tyrosinase and their mixtures.

4. The composition of claim 1 which may be used for determining the presence or absence of said nucleic acid amplification product utilizing assays specific for said reporter molecule, such that visual detection may be seen by the unassisted human eye.

5. A method of co-purifying one or more reporter molecules and the target product of a nucleic acid amplification to a binding matrix, wherein said reporter molecule, in the absence of the nucleic acid amplification product, has substantially no binding affinity for the binding matrix under the binding conditions used, and when the nucleic acid amplification product is present, then the nucleic acid amplification product and the reporter molecule both co-purify to the binding matrix, and the reporter molecule may be detected.

6. The method of claim 5, wherein the reporter molecule may be selected from the group consisting of alkaline phosphatase, glucose-6-phosphate dehydrogenase, catalase, horseradish peroxidase, glucose oxidase, glucose dehydrogenase, NADH oxidase, uricase, urease, creatininase, sarcosine oxidase, xanthine oxidase, creatinase, creatine kinase, creatine amidohydrolase, cholesterol esterase, cholesterol oxidase, glycerol kinase, hexokinase, glycerol-3-phosphate oxidase, lactate dehydrogenase, alanine transaminase, aspartate transaminase, amylase, lipase, esterase, gammaglutamyl transpeptidase, L-glutamate oxidase, pyruvate oxidase, diaphorase, bilirubin oxidase, laccase, tyrosinase and their mixtures.

7. The method of claim 5, wherein the reporter molecule may be detected by reacting with a substrate that produces a product in sufficient quantity that it is discernible to the unaided human eye.

8. The method of claim 5, wherein the reporter molecule and target nucleic acid copurify by adsorbing to a binding matrix.

9. The method of claim 5, wherein the reporter molecule is bound to a nucleotide sequence complementary to the nucleotide sequence of the target product.

10. The method of claim 5, wherein the binding matrix may be selected from the group consisting of cellulose, cellulose with functionally charged groups linked to cellulose, nitrocellulose, cellulose acetate, nylon, poly vinyl diflouride, pectin, silica, chemically modified silica, or combinations thereof.

11. The method of claim 5, wherein the binding solution comprises a polyalkyene glycol, 1,2 propane diol, 1,3 propane diol, glycerol, 1-thioglycerol, a salt, an alcohol, or combinations thereof.

12. The method of claim 5, wherein one or more reporter molecules is bound to a particle.

13. The method of claim 12, wherein the particle comprises a metal ion or a metal oxide that may be selected from the group consisting of iron, copper, gallium, cobalt, nickel, calcium, zinc, cadmium, silver, gold, hafnium, zirconium, titanium, palladium, platinum, aluminum, vanadium, lead, manganese, tin, ruthenium, and combinations thereof.

14. The method of claim 12, wherein the particle comprises cellulose, cellulose with functionally charged groups linked to cellulose, nitrocellulose, cellulose acetate, nylon, poly vinyl diflouride, pectin, pectin with functional groups attached, cellulose with positively charged groups attached, pectin with positively charged groups attached, silica, chemically modified silica, or combinations thereof.

15. The method of claim 12, wherein the particle is also the binding matrix.

16. The method of claim 12, wherein the particle is between 50 angstroms and 10,000 Angstroms in size.

17. A kit comprising a binding matrix, a binding solution, and a reporter molecule.

18. The kit of claim 17, wherein the binding solution may contain one or more reporter molecules, or the reporter molecules may be included separately.

19. The kit of claim 17, wherein said kit includes instructions for determining the presence or absence of a nucleic acid amplification product in a nucleic acid amplification reaction, wherein the binding matrix comprises one or more particles to which one or more reporter molecules has been bound thereto.

20. The kit of claim 17, wherein the binding matrix may comprise a particle to which one or more reporter molecules has been bound thereto.
